(19) United States
(12) Patent Application Publication
(10) Pub. No.: US 2022/0125859 A1
(43) Pub. Date: Apr. 28, 2022

(54) AGENTS AND METHOD FOR IMPROVIING GUT HEALTH

(71) Applicant: ZIVO Bioscience, Inc., Keego Harbor, MI (US)

(72) Inventors: Amy E. Steffek, Royal Oak, MI (US); William P. Pfund, Kalamazoo, MI (US); Andrew A. Dahl, Bloomfield Hills, MI (US)

(73) Assignee: ZIVO Bioscience, Inc., Keego Harbor, MI (US)

(21) Appl. No.: 17/465,457

(22) Filed: Sep. 2, 2021

Related U.S. Application Data
(60) Provisional application No. 63/073,805, filed on Sep. 2, 2020.

(51) Int. Cl. A61K 35/741; A61K 9/00; A61P 1/14; A23K 10/18; A23K 50/75; A23K 20/158

(52) U.S. Cl. CPC A61K 35/741; A61K 9/0056; A23K 20/158; A23K 10/18; A23K 50/75; A61P 1/14

(57) ABSTRACT

An improved agent and treatment method for a broad variety of diseases in both animals and humans are disclosed. The agent is an inventive treatment compound comprising a bacterial-based culture. The inventive treatment compound has a positive impact on the gut health of boilers from the earliest stage onward. These benefits are realized through several mechanisms, including improved gut morphology under disease stress. Healthier gut morphology and improved gut integrity result in improved nutrient uptake and growth benefits, both based on consumption of the disclosed inventive treatment compound. The downstream results are improvements in performance parameters related to gut health including altering gut microbes, feed conversion rates, and body weight gains among others. Use of the disclosed treatment compound has the further benefit of avoiding contamination during animal processing because of the reduction in the volume of pathogenic bacteria in the gut.

### CROSS REFERENCE TO RELATED APPLICATION

This application is a US. Non-provisional Patent Application of U.S. Provisional Patent Application No. 63/073,805, entitled “Agents and Method for Improving Gut Health,” filed Sep. 2, 2020, which is herein incorporated by reference in its entirety for all purposes.

### TECHNICAL FIELD

The present invention relates to the use of a bacteria-based compound for the improvement of gut health. More particularly, the present invention relates to a compound and the use of a compound such as that derived from a lipopolysaccharide (LPS) of Gram-negative bacteria that selectively modulates Toll-like receptors (TLRs) for the improvement of gut health in both animals and humans.

### BACKGROUND OF THE INVENTION

The overall health of the digestive tract of all animals, and specifically in poultry, is of critical concern in many industries. Poor digestive health results in lowered production rates and increased morality rates in production animals. Positive gut health is directly related to the overall performance, health and welfare of animals used in production. Gut health is itself highly complex, depending as it does on a variety of factors, including nutrition and immunology.

Negative gut health generally interferes with the normal conversion of animal feed into body weight gains. Gut morphology may be compromised by several factors, including disease and infection. Particularly susceptible to disease are the villi, surface area-expanding projections from the epithelial lining of the gut mucosa. Damage to gut morphology due to disease and infection compromise the ability of the intestine to absorb nutrients. In such a circumstance, efficient feed utilization drops, growth performance is jeopardized, and the risk of disease is increased.

The primary causative agents for gut disease include parasites and bacteria. Both agents take different courses in damaging gut health but both result in devastation to the animal as well as significant commercial loss.

The most common parasitic disease in poultry is coccidiosis. Caused by a protozoal parasite belonging to the genus Eimeria, coccidiosis may infect several locations in the intestine. Once introduced into the gut, the parasite quickly reproduces and, following a typical period of less than a week, causes damage to the intestinal mucosa and consequential loss of absorptive surface area. In poultry, a variety of coccidiosis-causing parasite species are known to exist, with each parasitizing different parts of the gut. The diseased birds suffer from an inability to absorb nutrients, in part, because of the morphologic change in the intestinal mucosa brought on by coccidiosis.

The most common bacterial disease in poultry is necrotic enteritis. Damage caused to the gut by coccidiosis may be a predisposing factor to the rapid onset of bacterial infection given that the intestinal mucosa is already compromised, leaving the animal susceptible to bacterial infection. It is thus very common for coccidiosis and necrotic enteritis to occur at the same time in a flock. An outbreak of necrotic enteritis in a flock may initially be overlooked as being little more than diarrhea and wet litter. Over time, typically by Day 35, the flock shows signs of compromised nutrient absorption in response to failing gut health.

The bacterium responsible for necrotic enteritis, Clostridium perfringens, is commonly found in animal litter, feces, feed, soil, and dust. It may also be found in the gut of healthy poultry in low levels. Two types of Clostridium perfringens, types A and C, are ordinarily the causes of necrotic enteritis in poultry. Toxins generated by these bacteria types are known to damage the small intestine and create liver lesions in poultry.

Bacterial infection in poultry not only puts the live animal at risk of injury, but also creates a potential risk during animal processing. Equipment used during poultry slaughter and processing may become contaminated from a bacteria-infected chicken, resulting in the contamination of poultry products intended for human consumption.

It is thus desirable to develop an agent and method of treatment for coccidiosis in animals, particularly in poultry, thereby strengthening gut health. Not only will improved gut health of the animal reduce or eliminate the incidence of coccidiosis, but, as a consequence, the incidence of bacterial infection and resulting diseases to an already weakened gut such as necrotic enteritis will also be reduced.

### SUMMARY OF THE INVENTION

The disclosed inventive concept provides an improved agent and treatment method for a broad variety of diseases in both animals and humans. The agent is an inventive treatment compound comprising a bacterial-based culture. Data suggest that the inventive treatment compound disclosed herein has a positive impact on the gut health of boilers from the earliest stage onward. Reference may be made to Applicants' co-owned and pending application U.S. Ser. No. 63/044,70, filed Jun. 26, 2020, for “Positive Latency Effects on Coccidiosis Prevention and Treatment Via Animal Feed,” which is herein incorporated by reference in its entirety for all purposes. These benefits are realized through several mechanisms, including improved gut morphology under disease stress. Healthier gut morphology and improved gut integrity result in improved nutrient uptake and growth benefits, both based on consumption of the disclosed inventive treatment compound. The downstream results are improvements in performance parameters related to gut health including altering gut microbes, feed conversion rates, and body weight gains among others.

Effective use of the disclosed treatment compound has the further benefit of avoiding contamination during animal processing because of the reduction in the volume of pathogenic bacteria in the gut of the broiler. The resulting improvement in sanitation reduces the likelihood of diseased animal products reaching consumers.

The mechanisms of action of the disclosed inventive compound and method are via immune system priming, a form of immune modulation, rather than a direct effect on pathogens, thus there is no risk of treatment resistance being developed. It is to be further noted that while the treatment compound and method of treatment disclosed herein are primarily directed to the improvement of gut health in poultry, the same compound and treatment methodology may effectively be applied to other animal species having similar conditions and issues.

### DESCRIPTION OF THE DRAWINGS

For a more complete understanding of this invention, reference should now be made to the accompanying figures. As set forth in the figures, the designation “CONTROL” refers to a control group of test subjects which received no additive ingredient in feed, the designation “CONROL+COCCI” refers to a group of test subjects from the control group infected with coccidiosis, the designation “COBAN+COCCI” refers to a group of test subjects which received the anti-coccidia drug Coban® (Elanco) and which were infected with coccidiosis, and “ALGAE+COCCI” refers to a group of test subjects which received the treatment composition of the disclosed inventive concept and which were infected with coccidiosis.

The accompanying figures are described as follows:

FIG. 1A is a graph illustrating test subject feed consumption data for Days 0 to 14;

FIG. 1B is a graph illustrating test subject feed consumption data for Days 0 to 21;

FIG. 1C is a graph illustrating test subject feed consumption data for Days 0 to 35;

FIG. 1D is a graph illustrating test subject feed consumption data for Days 0 to 42;

FIG. 2A is a graph illustrating test subject average body weight gain for Days 0 to 14;

FIG. 2B is a graph illustrating test subject average body weight gain for Days 0 to 21;

FIG. 2C is a graph illustrating test subject average body weight gain for Days 0 to 35;

FIG. 2D is a graph illustrating test subject average body weight gain for Days 0 to 42;

FIG. 3A is a graph illustrating test subject feed conversion data for Days 0 to 14;

FIG. 3B is a graph illustrating test subject feed conversion data for Days 0 to FIG. 3C is a graph illustrating test subject feed conversion data for Days 0 to 35;

FIG. 3D is a graph illustrating test subject feed conversion data for Days 0 to 42;

FIG. 4A is a graph illustrating test subject mortality for Days 0 to 14;

FIG. 4B is a graph illustrating test subject mortality for Days 0 to 21;

FIG. 4C is a graph illustrating test subject mortality for Days 0 to 35;

FIG. 4D is a graph illustrating test subject mortality for Days 0 to 42;

FIG. 5 is a graph illustrating test subject coccidia check score determined at Day 21;

FIG. 6A is a graph illustrating test subject lesion score determined at Day 21;

FIG. 6B is a graph illustrating test subject lesion score determined at Day 42;

FIG. 7A is a graph illustrating test subject ileum villi cell height on Day 21;

FIG. 7B is a graph illustrating test subject ileum villi cell height on Day 42;

FIG. 7C is a graph illustrating test subject ileum crypt depth on Day 21;

FIG. 7D is a graph illustrating test subject ileum crypt depth on Day 42;

FIG. 7E is a graph illustrating test subject ileum cell height to crypt depth ratio on Day 21;

FIG. 7F is a graph illustrating test subject ileum cell height to crypt depth ratio on Day 42;

FIG. 8A is a graph illustrating test subject ileum villi cell height on Day 42;

FIG. 8B is a graph illustrating test subject ileum crypt depth on Day 21;

FIG. 8C is a graph illustrating test subject ileum crypt depth on Day 42;

FIG. 8D is a graph illustrating test subject ileum cell height to crypt depth ratio on Day 21;

FIG. 8E is a graph illustrating test subject ileum cell height to crypt depth ratio on Day 42;

FIG. 8F is a graph illustrating test subject Salmonella cecum count on Day 21;

FIG. 8G is a graph illustrating test subject Salmonella cecum count on Day 42; and

FIG. 8H is a graph illustrating test subject Clostridium perfringens fecal count on Day 21.

### DETAILED DESCRIPTION OF THE PREFERRED EMBODIMENT

In the following description, various operating parameters and components are described for different constructed embodiments. These specific parameters and components are included as examples and are not meant to be limiting. Unless otherwise noted, all technical and scientific terms used herein are to be accorded their common meanings as would be understood by one having ordinary skill in the art.

The Compound Used in Treatment

The disclosed method of treatment preferably, but not absolutely, utilizes a compound generally derived from a lipopolysaccharide (LPS) of Gram-negative bacteria. By administering the compound early in broiler life, disease prevention and treatment via immune modulation are achieved. As used herein, the term “inhibitor” refers to a molecule that reduces or attenuates the activity induced by another molecule, receptor, cellular structure, or organ. By way of example, a compound that might block the LPS-dependent activation of TLRs, such as TLR4, present on the surface of a host immune cell would be regarded as an inhibitor of this particular pathway.

As used herein, “modulator” refers to an activator, an inhibitor, or both. Modulation may be the result of activity by at least one Toll-like receptor (TLR), such as TLR4 or possibly TLR2. As used herein, the term “inhibitor” refers to a molecule that reduces or attenuates the activity induced by another molecule. By way of example, a compound that might block the LPS-dependent activation of TLRs present on the surface of immune cells in humans and animals would be regarded as an inhibitor of this particular pathway.

As used herein, the term “algal culture” is defined as an algal organism and bacteria (one or more types) that grow together in a liquid medium. Unless expressly stated otherwise, the term “algal biomass” refers to the algal cells and bacterial cells (with the liquid culture medium removed). The “algal biomass” can be wet material or dried material.

Unless expressly stated otherwise, the term “algal supernatant” is defined as the culture medium in which the algal biomass is grown that contains excreted compounds from the algal biomass. Algal supernatant is obtained by growing algal biomass in culture medium for an appropriate length of time and then removing the algal and bacterial cells by filtration and/or centrifugation.

It is known that bacteria of the Variovorax genus and the Rhodobacter genus are metabolically versatile. Variovorax is a Gram-negative aerobic bacterium that can grow under a variety of conditions. It is part of the subclass Proteobacteria and is capable of metabolically utilizing several natural compounds generated by plants or algae. Rhodobacter can grow under a broad variety of conditions, utilizing both photosynthesis and chemosynthesis. Growth can also be achieved under both anaerobic and aerobic conditions. Rhodobacter sphaeroides represents a Gram-negative facultative bacterium and is a member of the α-3 subdivision of the Proteobacteria.

Embodiments of the compound used in the treatment of disease as set forth herein include one or more LPS/Lipid A compounds produced by Gram-negative bacterial strains for use as selective modulators of the TLR signaling pathway, such as the TLR4 pathway. The disclosed inventive concept involves any combination of three fundamental steps: (1) the Gram-negative bacteria produces LPS/Lipid A compounds; (2) the LPS/Lipid compounds modulate TLR4 activity through activation or inhibition; and (3) a downstream effect results in enhanced innate and adaptive immune processes, thereby aiding in the treatment of coccidiosis, necrotic enteritis, and other conditions related to gut inflammation.

In an embodiment, the LPS/Lipid A compounds used as selective modulators of the TLR4 signaling pathway are produced from a Variovorax paradoxus strain. The Variovorax paradoxus strain may be a naturally occurring strain found in an algal biomass. (As noted above in Paragraph [0015], biologically active byproducts [including either excreted products or structural components] may be found in the algal supernatant.) The algal biomass may comprise the algal species Klebsormidium flaccidum. More specifically, the algal biomass culture may comprise the algal strain Klebsormidium flaccidum, var. ZIVO.

In another embodiment, the LPS/Lipid A compounds used as selective modulators of the TLR4 signaling pathway are produced from a Rhodobacter sphaeroides strain. Extensive studies have been undertaken regarding the structure and function of Rhodobacter sphaeroides. More focused studies have examined the photosynthetic characteristics of Rhodobacter sphaeroides. While it is known that lipopolysaccharides from Rhodobacter sphaeroides are effective TLR4 antagonists in human cells that prevent TLR4-mediated inflammation by blocking LPS/TLR4 signaling, the inventors employed a testing methodology to address live growth performance parameters in poultry to arrive at the conclusion that an LPS compound derived from Rhodobacter sphaeroides proved effective as an alternative treatment for coccidiosis in poultry. Initial data suggested immune modulation by an LPS-like molecule, it was not until specific testing directed to Rhodobacter sphaeroides revealed the effectiveness of this bacterium in the treatment of disease, such as in the treatment of coccidiosis in poultry. Research further showed that combining a compound that acts as a TLR4 inhibitor in human in vitro assays with an activator of TLR2 (such as lipoproteins from Gram-negative bacteria) provides an anti-coccidiosis effect.

Accordingly, embodiments of the compound used in the treatment of disease according to the present disclosure are directed to one or more LPS/Lipid A compounds produced by a Gram-negative bacterial strain of the group Variovorax or the group Rhodobacter for use as selective modulators of the TLR signaling pathway. A specific embodiment of the disclosed inventive concept is directed to the use of an LPS/Lipid A compound used as a selective modulator of the TLR4 signaling pathway produced from the Variovorax paradoxus strain and the Rhodobacter sphaeroides strain.

The LPS/Lipid A compound employed herein may be obtained from the Variovorax paradoxus strain and/or the Rhodobacter sphaeroides strain by any suitable method, but in specific embodiments they are extracted using standard multi-step LPS extraction protocols, such as: (1) extracting freeze-dried bacteria with a solution of phenol/guanidine thiocyanate and collecting the water layer for freeze-drying; (2) resolubilizing the freeze-dried fraction in water; (3) ultrafiltration of the solubilized fraction to remove low molecular weight substances and salts; (4) affinity purifying the high-molecular weight fraction using a polymyxin B resin column such as Affi-prep polymyxin matrix material (Bio-Rad), from which an active fraction is eluted with 1 deoxycholate and, optionally; (5) performing additional purification using size-exclusion chromatography.

In some examples, multiple types of LPS extraction protocols are employed to obtain an LPS compound from the bacteria, and extraction procedures may be performed more than once. Once the LPS compound is extracted and purified from the bacteria, the Lipid A fraction may be prepared by acid hydrolysis or other suitable technique.

In some examples, analysis of the structure of the LPS compound is performed using routine methods in the art, including using mass spectrometry, gas chromatography, or both. In an embodiment, results of liquid chromatography analysis of the LPS isolated from the Variovorax paradoxus strain showed the presence of both hydroxy-decanoic and hydroxy-octanoic fatty acids on the lipid A moiety. In another embodiment, results of gas chromatography-mass spectrometry (GC-MS) analysis of the LPS isolated from the Variovorax paradoxus strain showed that the main saturated fatty acid is lauric acid, with one or two molecules per lipid A structure.

The one or more LPS/Lipid A compounds derived from Gram-negative bacterial strains, such as Variovorax paradoxus or Rhodobacter sphaeroides, may selectively modulate the TLR4 signaling pathway to alter inflammatory responses and to improve immune health in a variety of uses and applications. In an embodiment, the LPS/Lipid A compound derived from Variovorax paradoxus or Rhodobacter sphaeroides may be incorporated within an algal-based feed ingredient to improve gut health of poultry.

The disclosed LPS/Lipid A compound derived from Variovorax paradoxus or Rhodobacter sphaeroides may be used to improve the health of poultry through a variety of mechanisms. For example, the LPS/Lipid A compound may protect against internal inflammation in poultry by negatively regulating inflammatory mediators via the downregulation of TLR4 expression and the downstream inhibition of NF-kappa B activation in a typical inflammatory cascade. In another example, the LPS/Lipid A compound may inhibit the activation of TLR4 in poultry by interfering with cysteine residue-mediated receptor dimerization. In yet another example, the LPS/Lipid A compound may inhibit the ability of non-infectious and infectious stimuli to interact with TLR4 and trigger a pro-inflammatory response, thereby improving poultry gut integrity. In a further example, the LPS/Lipid A compound may modulate TLR4 through either ligand-dependent or ligand-independent activation. As another example, the LPS/Lipid A compound may act in concert with other TLR agonists to provide a heightened immune response, while reducing the metabolic costs to the host.

Study

A study was undertaken to determine the response and efficacy of a dried algal biomass feed ingredient incorporated at a specific amount into a commercial-type corn-soybean diet and fed to floor-pen raised broilers. The study was undertaken over a 42-day period, from Day 0 to Day 42. Particularly, the treatment compound is fresh water algal biomass containing Gram-negative bacteria provided as animal feed in combination of a feed additive, such as soy oil, preferably though not exclusively at a ratio of two parts soil oil to one part algal biomass. Once the biomass and feed additive are combined to the preferred premix level, the combined batch is poured or administered evenly into a ribbon mixer containing finished feed. The combined batch is preferably provided in an amount of between about 0.5 lbs. per ton and about 11.0 lbs. per ton of finished feed and is more preferably though not exclusively provided in an amount of about 3.5 lb/ton of feed (0.175% of the feed ration) with good efficacy without being wasteful. In general, treatment using the disclosed inventive treatment compound is around 700 mg per bird per the 42 day period.

The animals were raised under a disease challenge environment (cocci-challenge+built-up litter). The disclosed inventive treatment compound in the form of a dried algal biomass feed ingredient was given to a cocci-inoculated group (ALGAE+COCCI) that was compared to a cocci-inoculated control group (CONTROL+COCCI) that received no feed additive ingredient, a non-inoculated control group (CONTROL) that received no feed additive ingredient, and a cocci-inoculated group that received the anti-coccidia drug Coban® (Elanco) in feed at 90 g/ton (COBAN+COCCI).

To determine whether the feed pelleting process, which involves heat, altered the test feed ingredient in terms of the study endpoints, each of the four groups were fed diets in either MASH or PELLET form, for a total of eight treatment groups. Birds were evaluated in terms of physical live performance and digestive health from 0 to 42 days of age. Results were analyzed for differences among treatment groups (CONTROL, CONTROL+COCCI, COBAN+COCCI, ALGAE+COCCI) and by feed type (MASH vs. PELLETS).

Study—Treatment Method

A total of 5,500 mixed sex broiler chicks were obtained within twelve hours of hatching from fecal contaminated flocks at a commercial hatchery on Day 0 (hatch and placement day). A number of mixed-sex broiler chicks (50:50 sex ratio) were randomly assigned on Day 0 by individual weights to one of several test group pens, each with replicates. Only antibiotic-free birds were sourced, and no coccidiosis vaccine was administered at the hatchery or at any time during the study. Chicks were evaluated upon receipt for signs of disease or other complications that could affect study outcome. Weak birds were humanely sacrificed. Birds were not replaced during the study.

Following examination, chicks were weighed and allocated to pens for the various treatment groups using a randomized block design. Weight distribution across the treatment groups was assessed prior to feeding by comparing the individual test groups' standard deviations of the mean against that of the control group. Weight distribution across the groups was considered acceptable for this study when differences between control and test groups were within one standard deviation.

Treatment Groups—Treatment groups, the levels of test material, the number of replicates, the number of bird replicates, and the type of feed (mash vs. pellets) were established as follows.

| | | Test | | | |
|---|---|---|---|---|---|
| | | Material | | Birds per | Feed |
| | Treatment Groups | Level | Replicates | Replicate | Type |
| 1 | CONTROL (NO cocci- | NO | 12 | 52 | Mash |
| | challenge, NO feed | | | | |
| | additive) | | | | |
| 2 | CONTROL + COCCI | NO | 12 | 52 | Mash |
| | (YES cocci-challenge, | | | | |
| | NO feed additive) | | | | |
| 3 | COBAN + COCCI (YES | 90 g per ton | 12 | 52 | Mash |
| | cocci-challenge, YES | of finished | | | |
| | feed additive Coban) | feed | | | |
| 4 | ALGAE + COCCI (YES | 3.50 pounds | 12 | 52 | Mash |
| | cocci-challenge, YES | per ton of | | | |
| | feed additive dried algae | finished feed | | | |
| | biomass) | | | | |
| 5 | CONTROL (NO cocci- | NO | 12 | 52 | Pellets |
| | challenge, NO feed | | | | |
| | additive) | | | | |
| 6 | CONTROL + COCCI | NO | 12 | 52 | Pellets |
| | (YES cocci-challenge, | | | | |
| | NO feed additive) | | | | |
| 7 | COBAN + COCCI (YES | 90 g per ton | 12 | 52 | Pellets |
| | cocci-challenge, YES | of finished | | | |
| | feed additive Coban) | feed | | | |
| 8 | ALGAE + COCCI (YES | 3.50 pounds | 12 | 52 | Pellets |
| | cocci-challenge, YES | per ton of | | | |
| | feed additive dried algae | finished feed | | | |
| | biomass) | | | | |

All birds received nutritionally adequate diets as either mash or pellets. Birds were fed their respective treatment diets ad libitum from day of hatch to 42 days of age, the typical average market age of broiler chickens in US. Birds were raised on built-up litter to further mimic stress conditions typically experienced in poultry production.

Diets were weighed at the beginning of each formulation period and fed in three phases: Starter diet (0-21 days of age), Grower diet (22-35 days of age) and Finisher diet (36-42 days of age). Diets were fed for the entire study duration as either mash or as pellets (with pellets served as crumbles on days 0-21). All diets were offered ad libitum without restrictions to full-fed consumption, except for an 8-hour fasting period for cocci-inoculated birds prior to cocci-challenge on Day 7 when all birds in the challenged groups received oocyst-inoculated feed containing a mixture of Eimeria acervulina, Eimeria maxima, and Eimeria tenella. Dietary requirements for protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in poultry production.

Throughout the study, birds were observed at least three times daily for overall health, behavior, and evidence of toxicity. Pens were monitored for environmental conditions, including temperature, lighting, water, feed, litter condition, and unanticipated house conditions/events. Pens were checked daily for mortality. Examinations were performed on all broilers found dead or moribund. Mortalities were recorded (date and weight) and examined (both internal and external body mass).

Cocci-Challenge Model—On Day 7, all birds in the challenged groups received oocyst-inoculated feed containing a mixture of Eimeria acervulina, Eimeria maxima, and Eimeria tenella. Adequate feed was precisely weighed and provided to birds to consume at the rate of 100% fill-capacity on average. Prior to the challenge, all cocci-inoculated birds were starved for eight hours. Inoculated feed was provided to the birds. Following a specific time, all remaining inoculated feed was removed and weighed to assure equal consumption per pen and per bird. The quantity of feed (both placed and withdrawn) was recorded on each pen's feed record.

Study Evaluation

Differences between groups were evaluated at P<0.05, employing Treatment x Replicate RCB (Randomized Complete Block) design. The control group for comparison was the PELLET-fed CONTROL group (no cocci-inoculation, no feed additive), pairing each treatment type (i.e., both mash and pelleted feeds were employed for each of 4-treatment types). All data points were analyzed at the 5% level of probability, including composite weighted average of entire pen, feed:gain and mortality. Birds were evaluated in terms of physical live performance and digestive health from 0 to 42 days of age. Results were analyzed for differences among treatment groups (CONTROL, CONTROL+COCCI, COBAN+COCCI, ALGAE+COCCI) and by feed type (MASH vs. PELLETS).

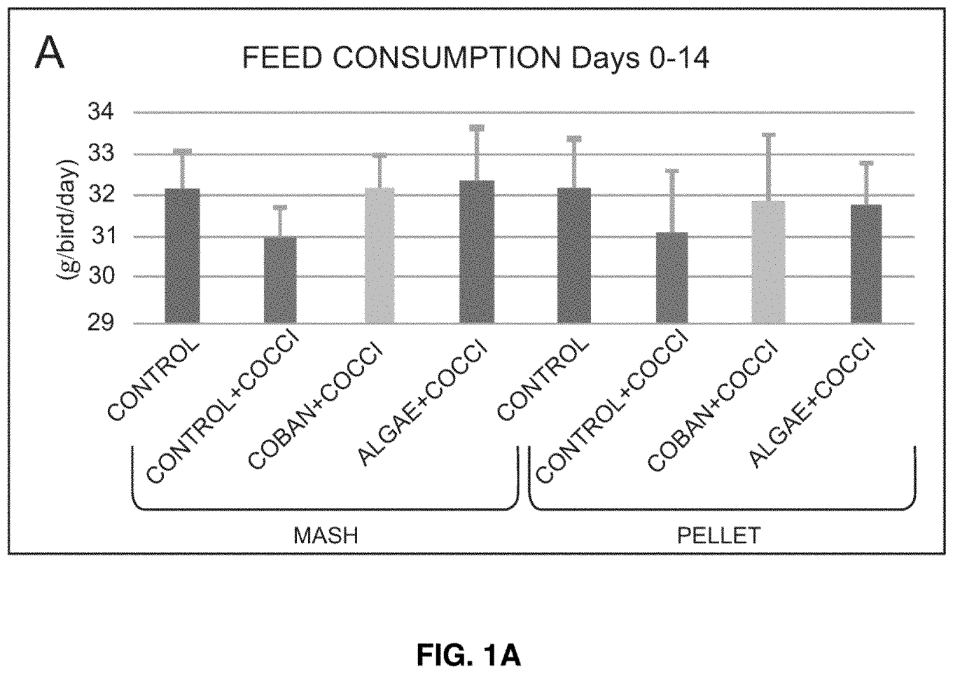

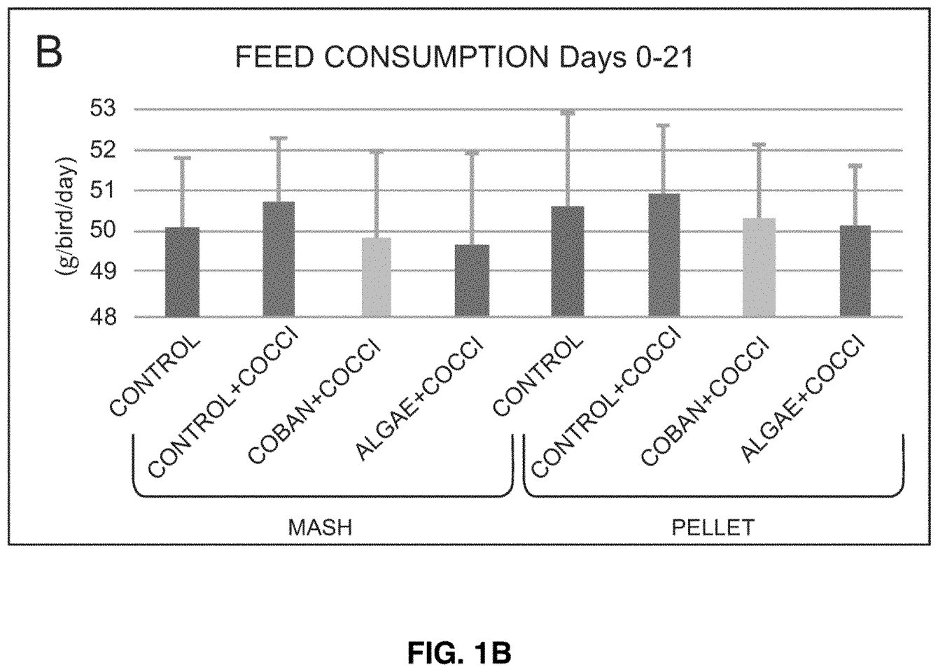

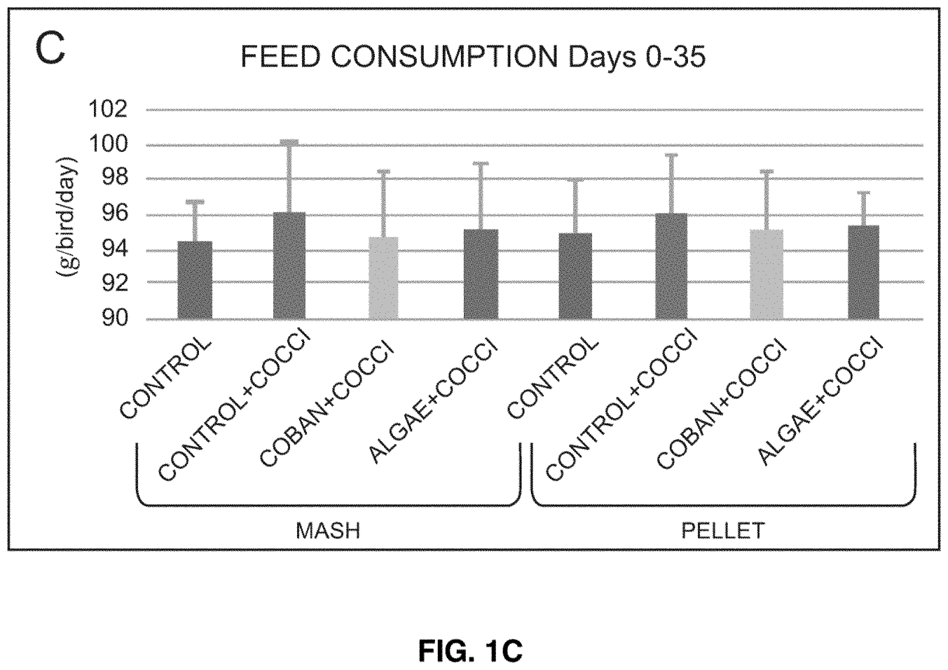

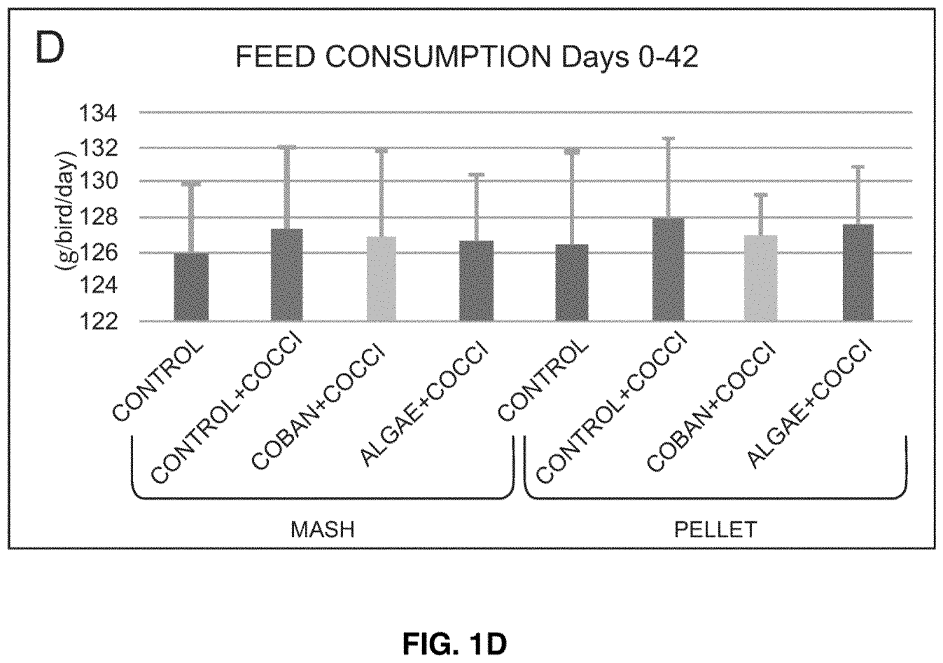

Live Performance Evaluation—Live performance parameters were recorded weekly throughout the study. The disease challenge environment (cocci-challenge+built-up litter) was employed effectively, as evidenced by the fact that the CONTROL group outperformed the CONTROL+COCCI group for weight gain, feed efficiency, and mortality across all age ranges (Days 0-14, 0-28, 0-35, and 0-42) in both the MASH and PELLET fed segments. Feed consumption is illustrated in FIG. 1A (Days 0-14), 1B (Days 0-21), 1C (Days 0-35), and 1D (Days 0-42). Feed consumption was significantly decreased in the CONTROL+COCCI group compared to CONTROL from Days 0-14 (seven days after cocci inoculation) in both the MASH and PELLET fed segments, further indicating a successful challenge model. After Day 14, there were no significant differences in feed consumption for any of the groups.

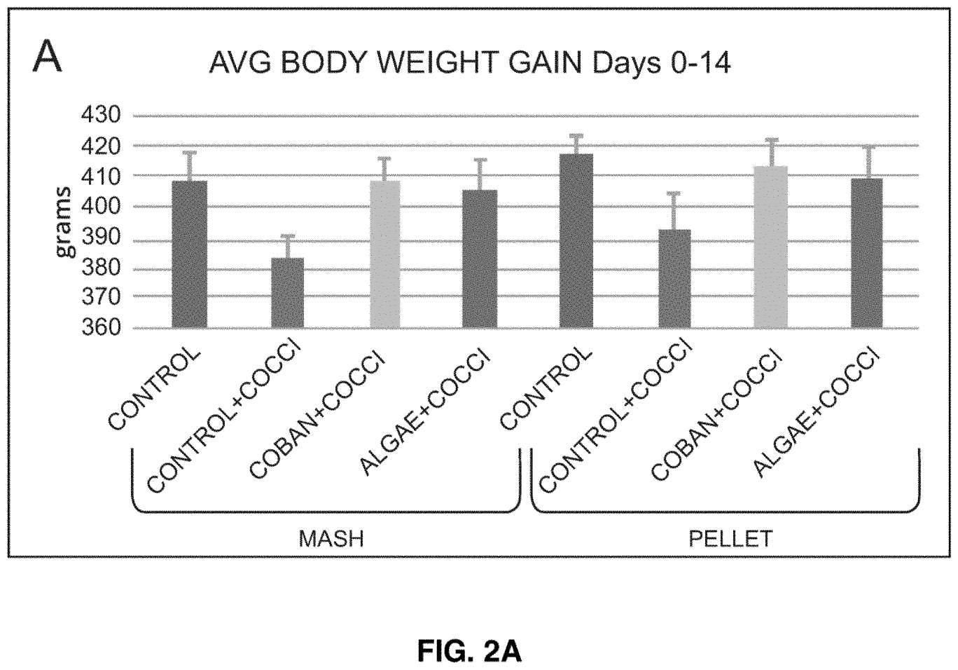

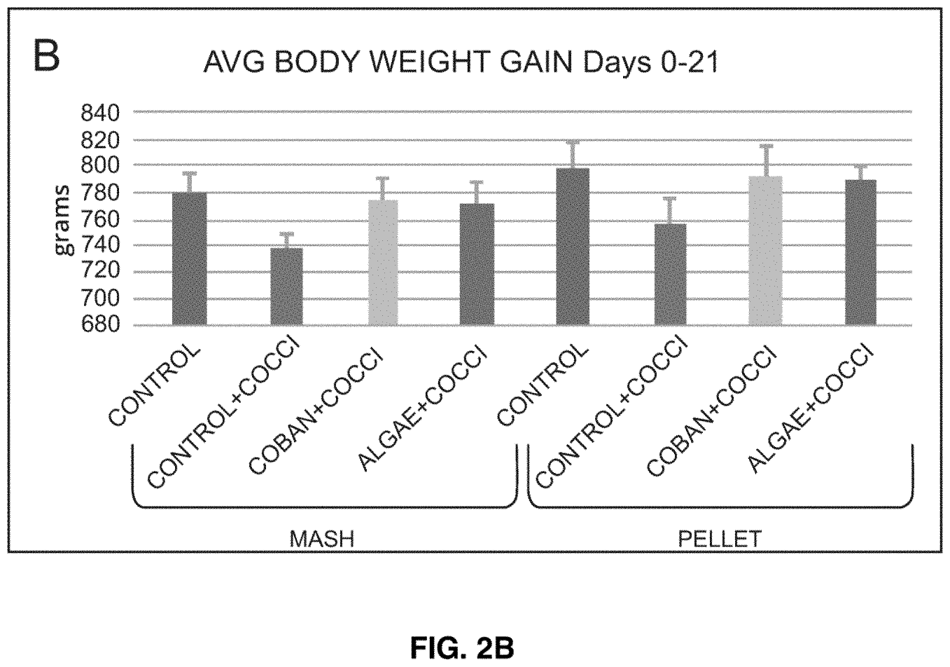

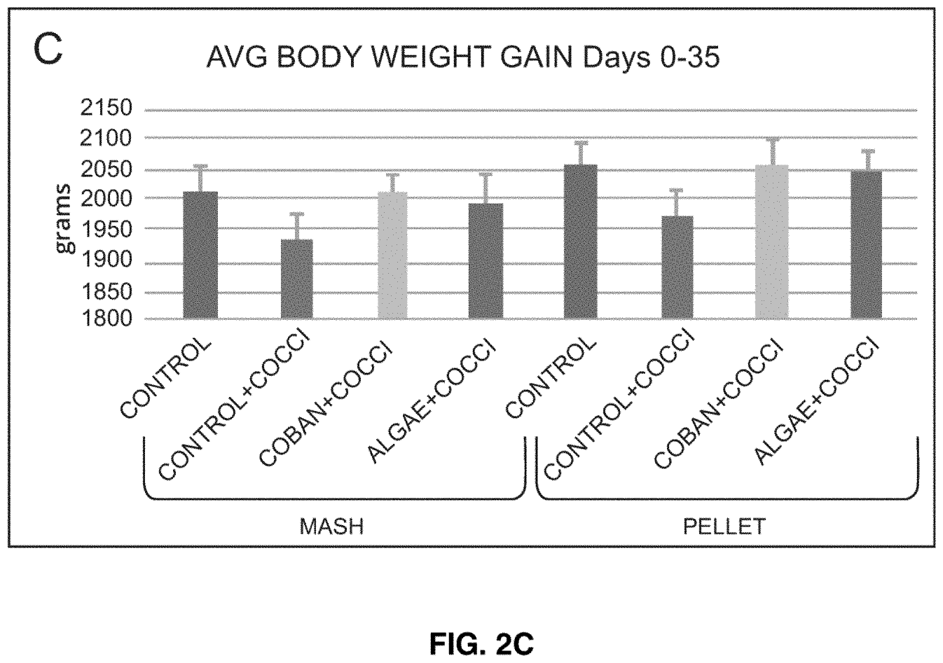

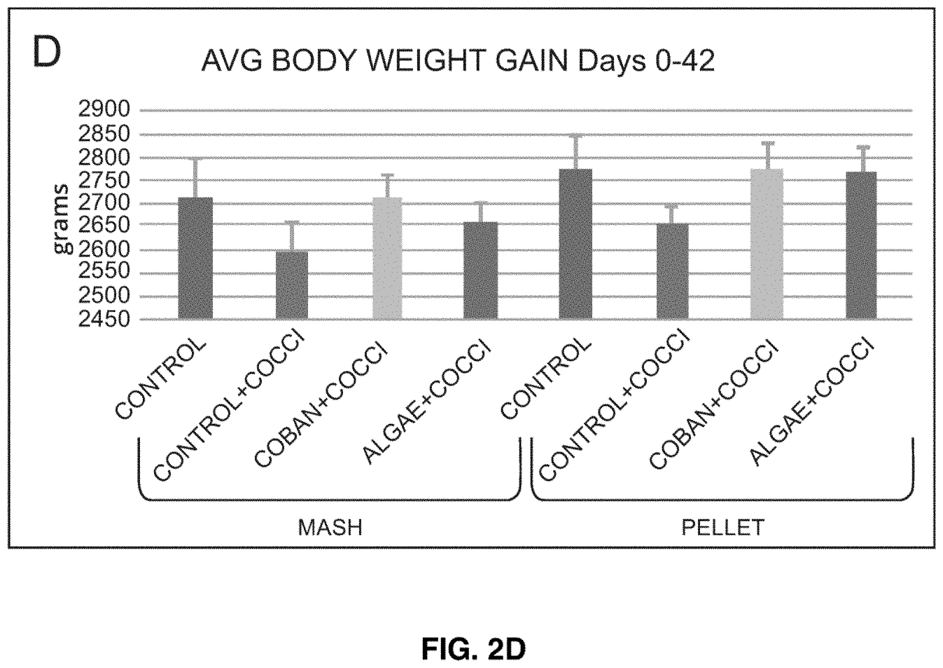

Body Weight Evaluation—Individual weights were recorded on Days 0, 14, 21, and 35 and Day 42 of the study and are set forth in FIGS. 2A-2D. On Day 0, there were no significant differences in average body weight by treatment group. Across all age periods, the average body weight and average body weight gain in the ALGAE+COCCI group was significantly increased compared to the CONTROL+COCCI group in both the MASH and PELLET fed segments. From Days 0-14, there were no statistical differences in body weight and body weight gain in the ALGAE+COCCI, COBAN+COCCI, and CONTROL groups in the MASH fed segment and they were all significantly improved compared to the CONTROL+COCCI group. In the PELLET fed segment, there were no differences in body weight and body weight gain from Days 0-14 in the ALGAE+COCCI and COBAN+COCCI groups. Across Days 0-21 and 0-35, there were no statistical differences in the body weight and body weight gain in the ALGAE+COCCI group compared to the CONTROL group in either the MASH or PELLET fed segments. From Days 0-42, there were no statistical differences in the body weight gain of the ALGAE+COCCI group compared to the CONTROL group in the MASH or PELLET fed segments or in body weight in the mash segment, while in the PELLET fed segment, there were no differences in body weight for ALGAE+COCCI and COBAN+COCCI groups.

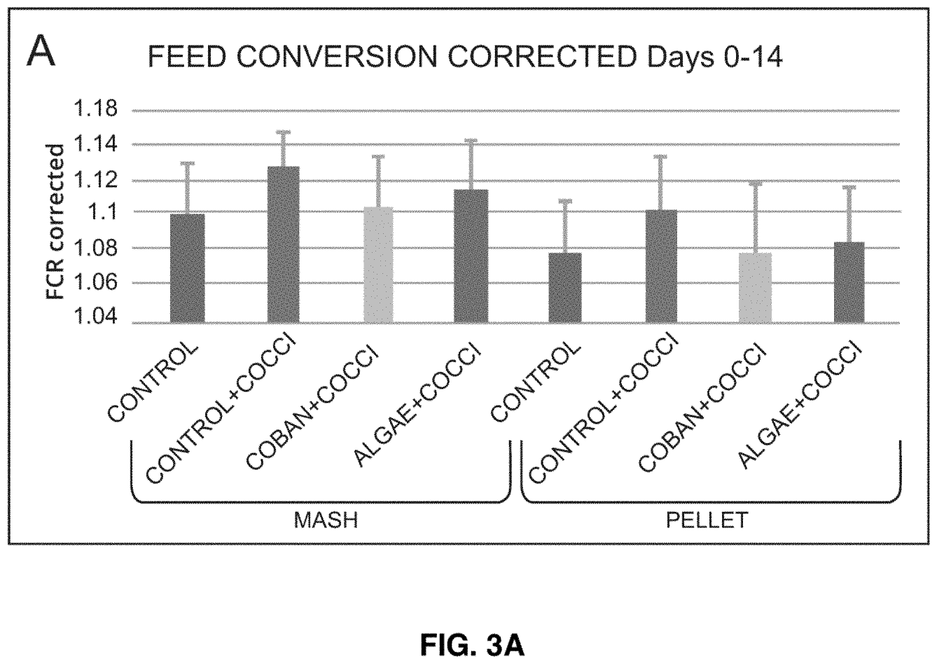

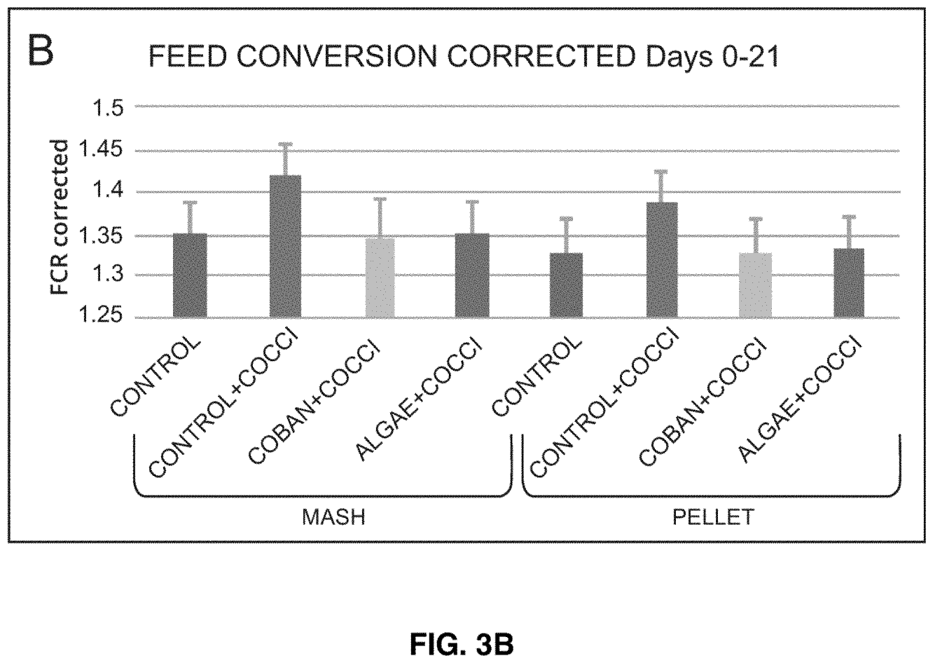

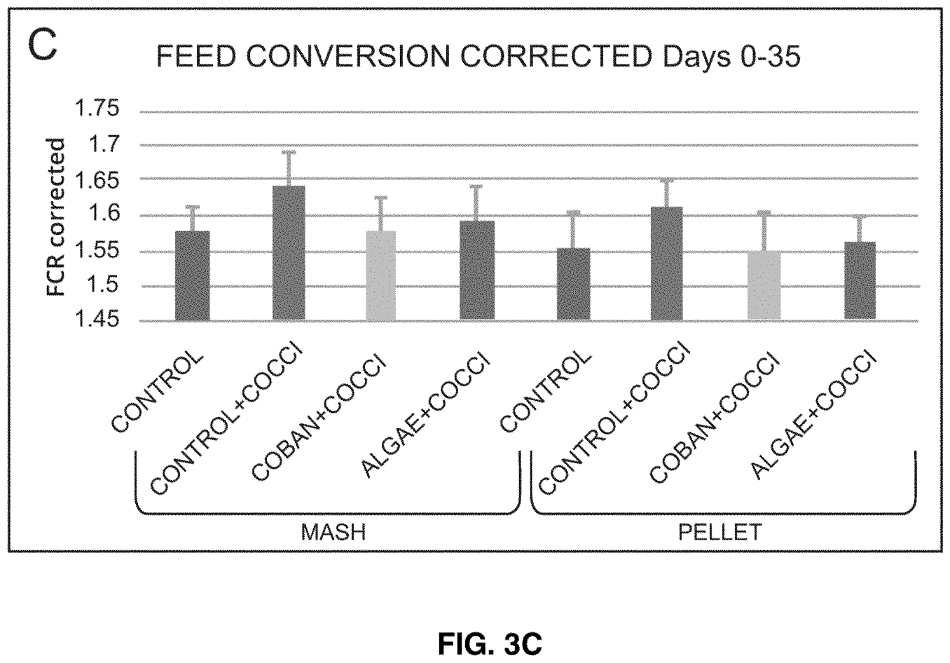

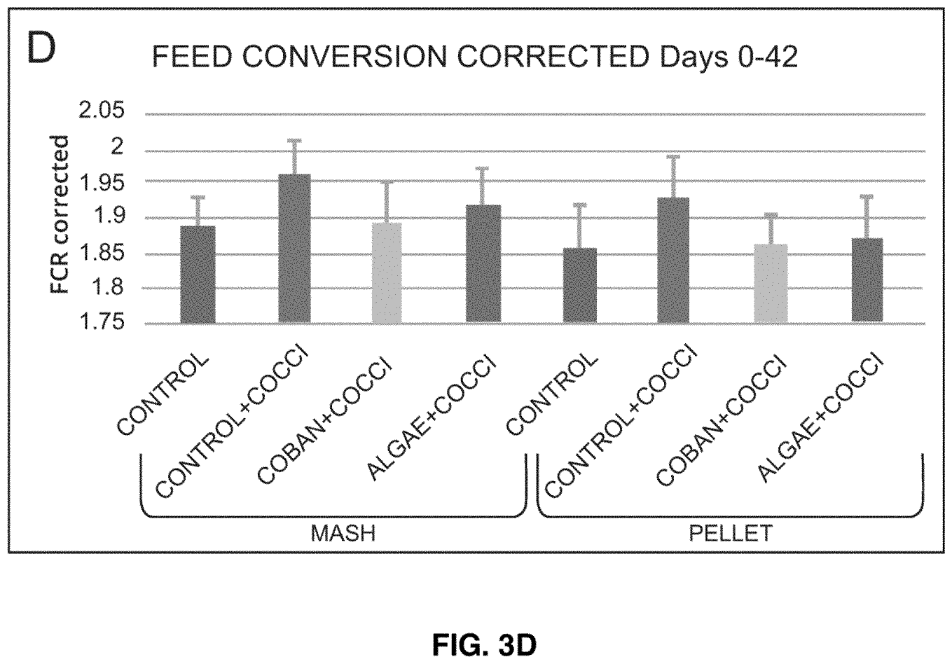

Feed Consumption/Feed Conversion—Individual Feed Consumption (g per bird per day) was measured and reported for Days 0-14, 0-21, 0-35 and 0-42. Feed conversion (corrected) data are illustrated in FIG. 3A (Days 0-14), 3B (Days 0-21), 3C (Days 0-35), and 3D (Days 0-42). As shown in the figures, the only differences in feed consumption among the groups occurred from Days 0-14 when the CONTROL+COCCI had a statistically significant decrease in feed consumption compared to all other groups in both the MASH and PELLET fed segments. The adjusted feed conversion ratio (FCR) was calculated for Days 0-14, 0-21, 0-35 and 0-42. From Days 0-14 there were no significant differences in FCR of the ALGAE+COCCI group compared to the other groups in either the MASH or PELLET fed segments. From Days 0-21 and 0-35, the FCR of the ALGAE+COCCI group was significantly improved compared to the CONTROL+COCCI group in both the MASH and PELLET fed segments and there was no difference compared to the CONTROL group. Over the course of the entire study (Days 0-42), there was no difference in the FCR of the ALGAE+COCCI and CONTROL groups in the PELLET fed segment, while there were no differences in ALGAE+COCCI compared to any of the groups (including CONTROL+COCCI) in the MASH fed segment.

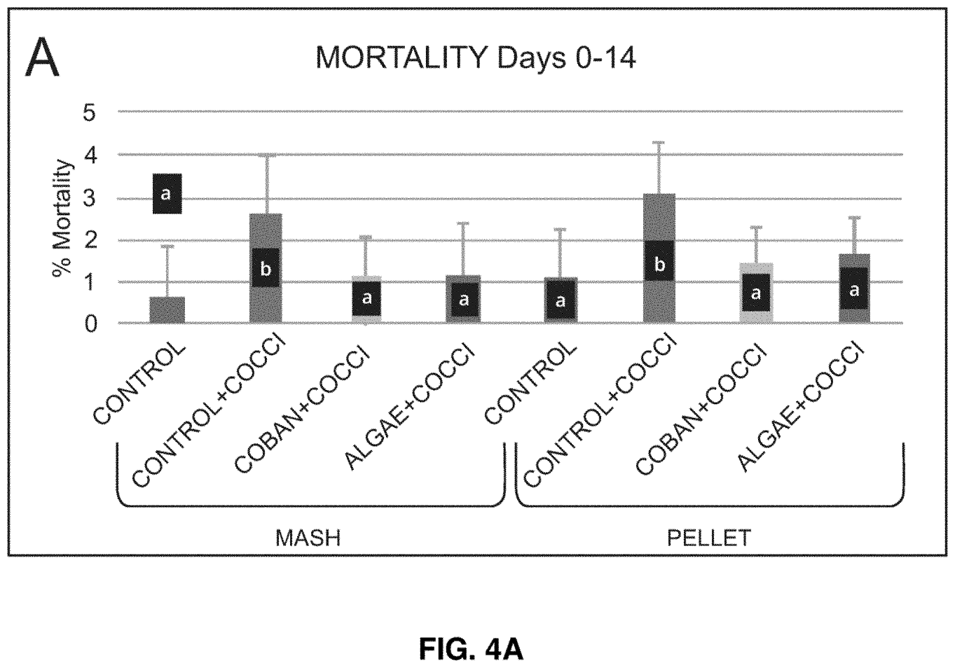

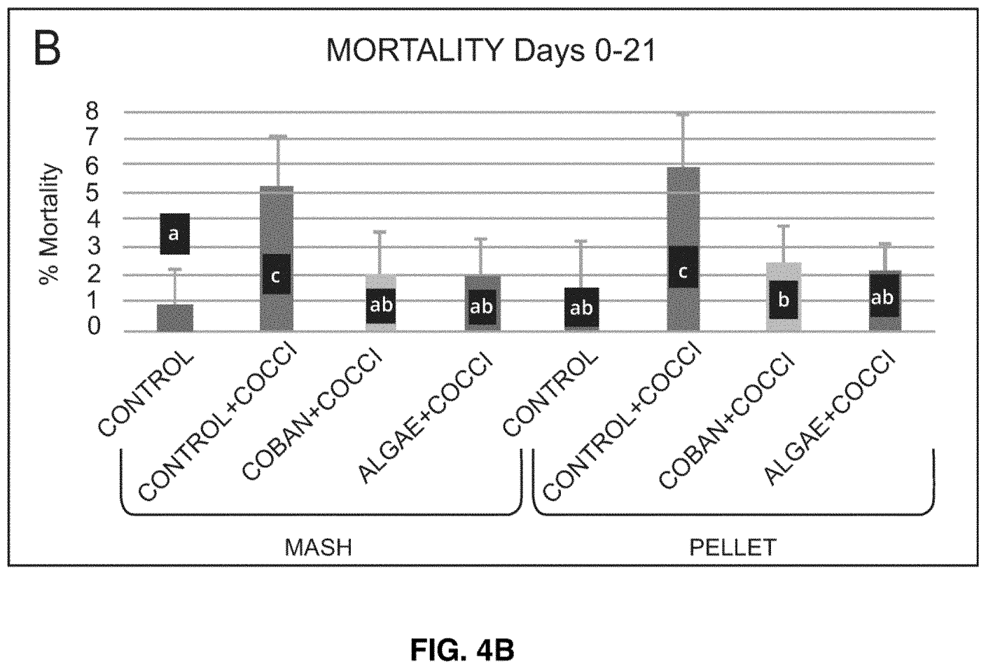

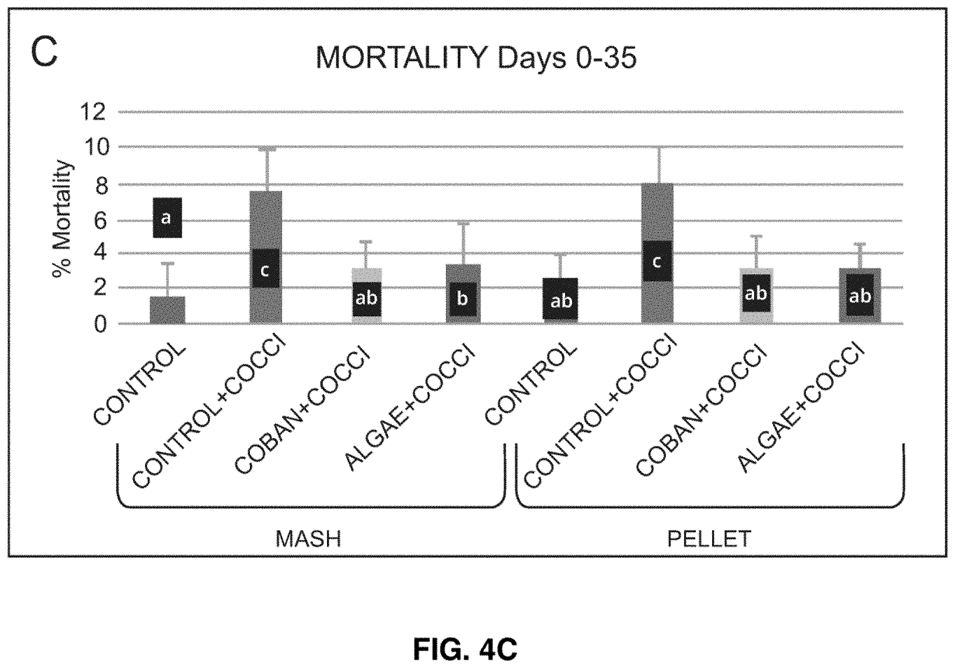

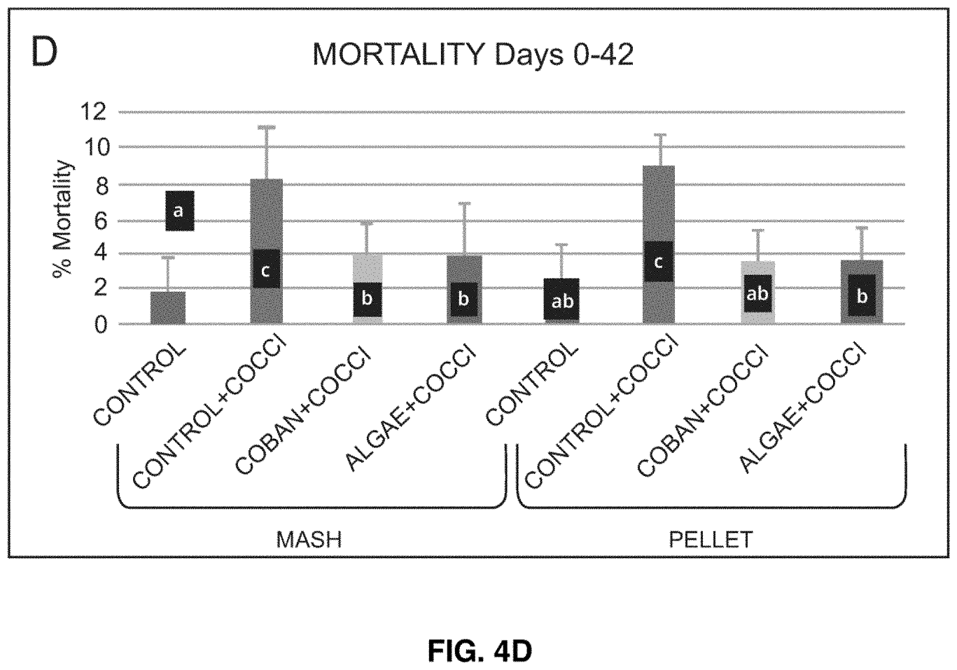

Mortality—As shown respectively in FIGS. 4A-4D, mortality was calculated for Days 0-14, 0-21, 0-35 and 0-42. Across all age periods, the % mortality in the ALGAE+COCCI treatment group was significantly decreased compared to the CONTROL+COCCI group in both the MASH and PELLET fed segments. For Days 0-14 and 0-21, there were no statistical differences in the ALGAE+COCCI group % mortality compared to the CONTROL group in either the MASH or PELLET fed segments. In fact, from Days 0-21, the ALGAE+COCCI group outperformed the COBAN+COCCI group in the PELLET fed segment in decreasing mortality. From Days 0-35 and 0-42, there were no statistical differences in the ALGAE+COCCI group % mortality compared to the COBAN+COCCI group in the MASH fed segment and no statistical differences compared to the CONTROL group in the PELLET fed segment.

Digestive Health—Strain-specific enumerations by gut area from 4-birds per pen/rep (12 reps per treatment) were performed for predominate E. acervulina in the loop of the small intestine, predominate E. maxima in the jejunum, and predominate E. tenella in the ceca. Samples were collected on Days 21 and 42. In both the MASH and PELLET fed segments, ALGAE+COCCI had a significantly lower Coccidia Check Score compared to CONTROL+COCCI. In the MASH fed segment, there were no differences in ALGAE+COCCI and COBAN+COCCI groups. In the PELLET fed segment, the ALGAE+COCCI group had a significantly lower score compared to the COBAN+COCCI group but did not reach levels of the CONTROL group.

Improved Gut Integrity—Data

As set forth above, treatment of animals, particularly poultry, using the disclosed treatment compound improves overall gut health. This is evidenced by the reduced number of lesions identified in the intestinal lining of subject birds. The following data disclose the effect of feed including a dried algal biomass in the form of the disclosed treatment compound on live performance of broilers raised under a disease challenged environment.

Graph—Coccidia Check Score—Strain-specific enumerations by gut area from 4-birds per pen/rep (12 reps per treatment) were performed for predominate E. acervuline in the loop of the small intestine, predominate E. maxima in the jejunum, and predominate E. tenella in the ceca. These data are reported as a 0 to 4 score, with the following rankings identified as:

0=No oocysts were found in the intestinal sample.
1=(+) Low level Coccidiosis close to a ‘natural’ uninoculated infection.

### 2=(++) Moderate Coccidiosis

### 3=(+++) Severe Coccidiosis.

4=(++++) Extremely severe Coccidiosis.

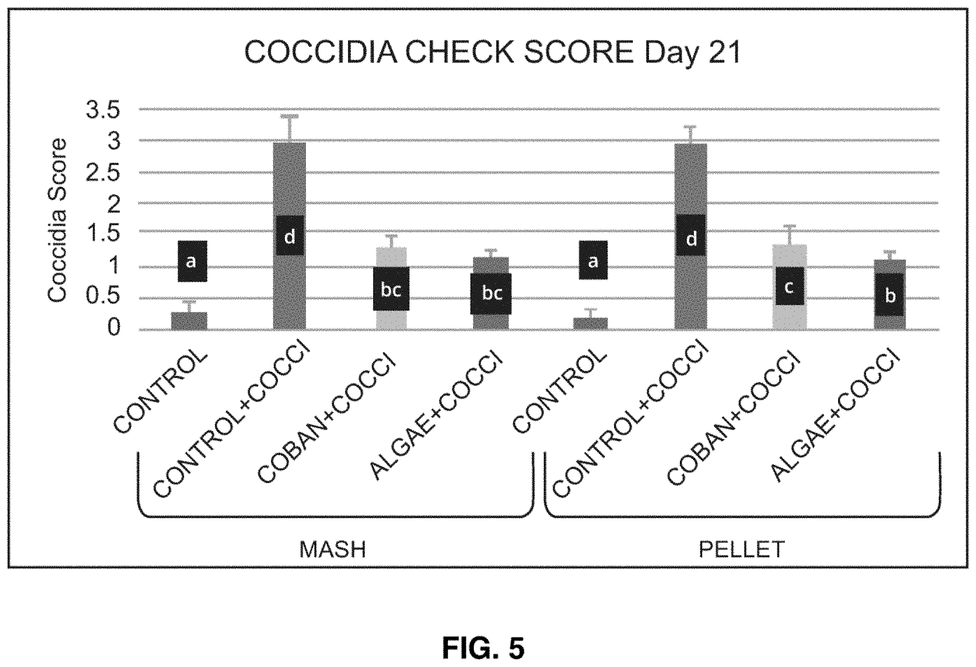

Samples were collected on Days 21 and 42, however, due to post-collection processing issues the Day 42 samples were not suitable for analysis. Thus, only Day 21 data are reported as set forth in FIG. 5. In both the MASH and PELLET fed segments, ALGAE+COCCI had a significantly lower Coccidia Check Score compared to CONTROL+COCCI. In the MASH fed segment, there were no differences in ALGAE+COCCI and COBAN+COCCI groups. In the PELLET fed segment, the ALGAE+COCCI group had a significantly lower score compared to the COBAN+COCCI group but did not reach levels of the CONTROL group.

Graphs—Lesion Scoring—Gross necropsy and lesion scoring were performed on Days 21 and 42. Birds were selected, sacrificed, weighed, and examined for the presence and degree of coccidia lesions and the amount of intestinal gut lining sluffing. ceca damage scores were assessed and recorded.

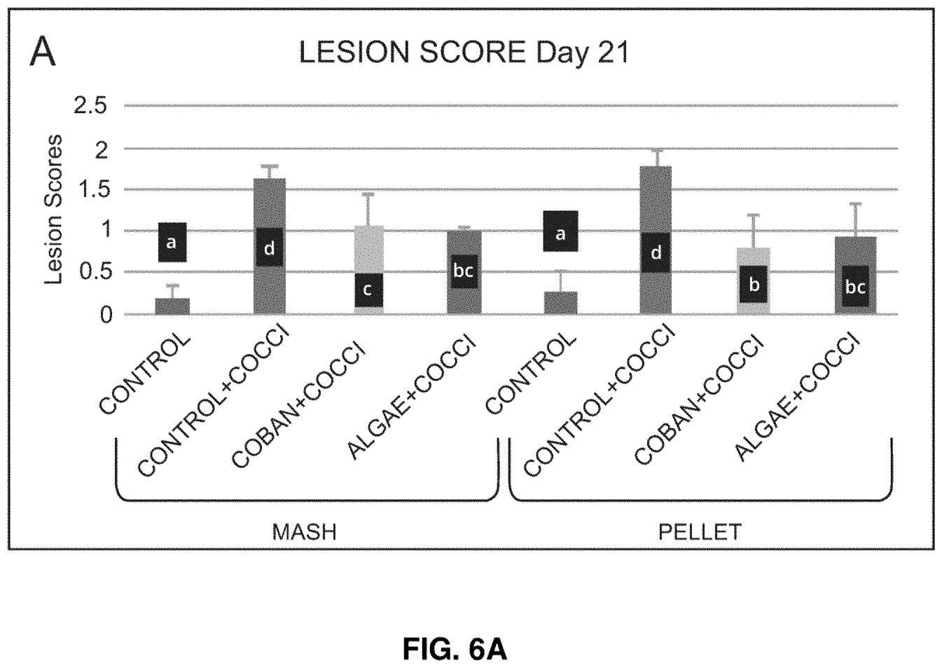

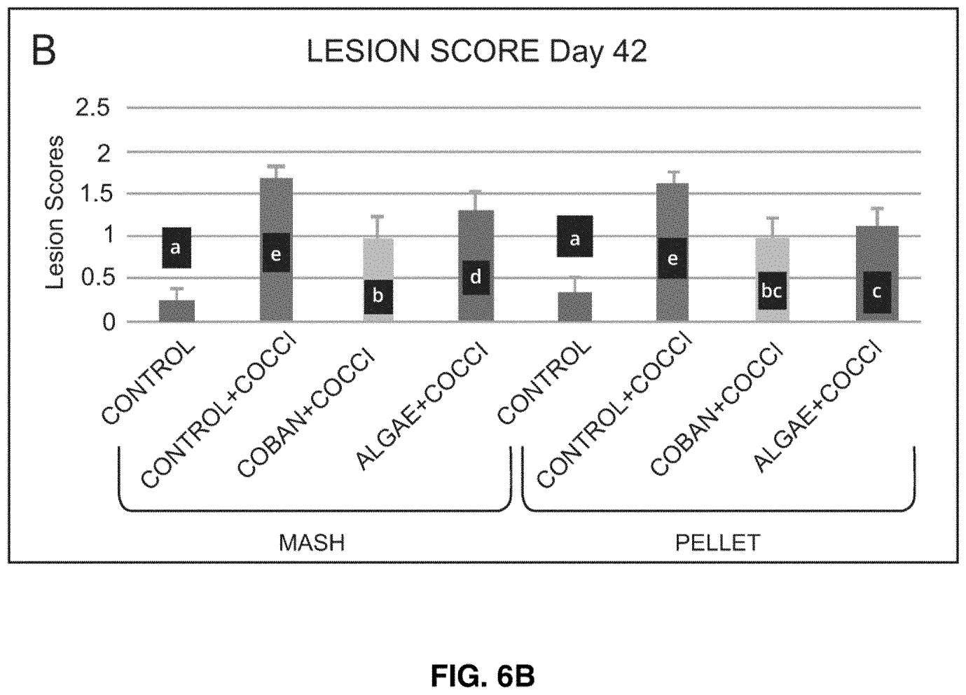

As illustrated in FIGS. 6A and 6B respectively, lesion scores in the ALGAE+COCCI treatment group were significantly decreased compared to the CONTROL+COCCI group in both the MASH and PELLET fed segments at both time points. On Day 21, there were no differences in the ALGAE+COCCI and COBAN+COCCI groups in both the MASH and PELLET fed segments. On Day 42, this was true only in the PELLET fed segment.

ILEUM VILLI CHARACTERISTICS—Ileum villi cell height, crypt depth, and the villus height to crypt depth ratio were taken, calculated and reported from the ileum area on Days 21 and 42.

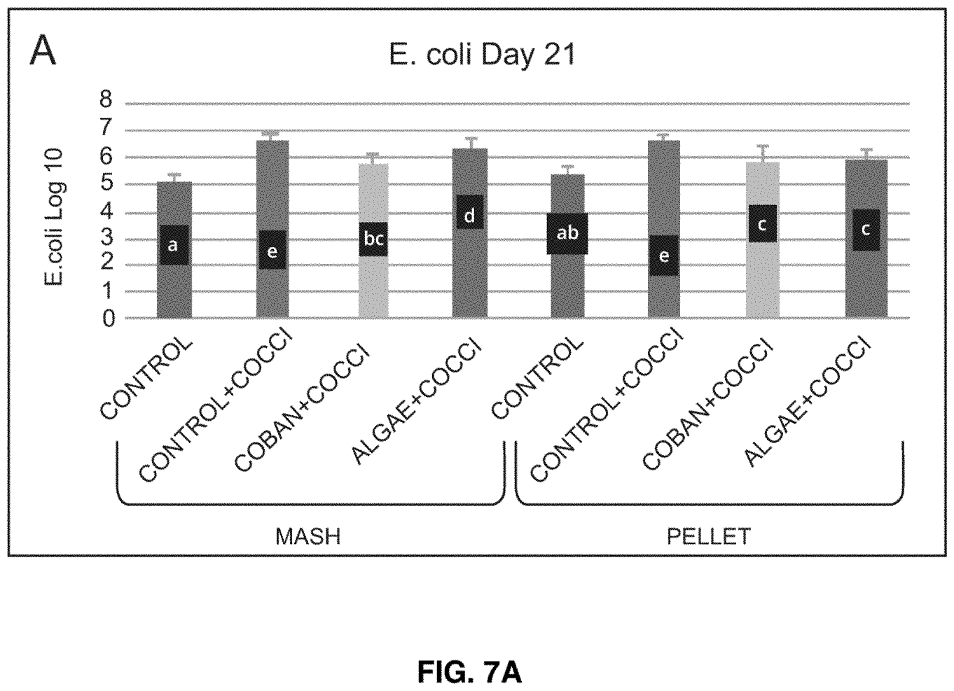

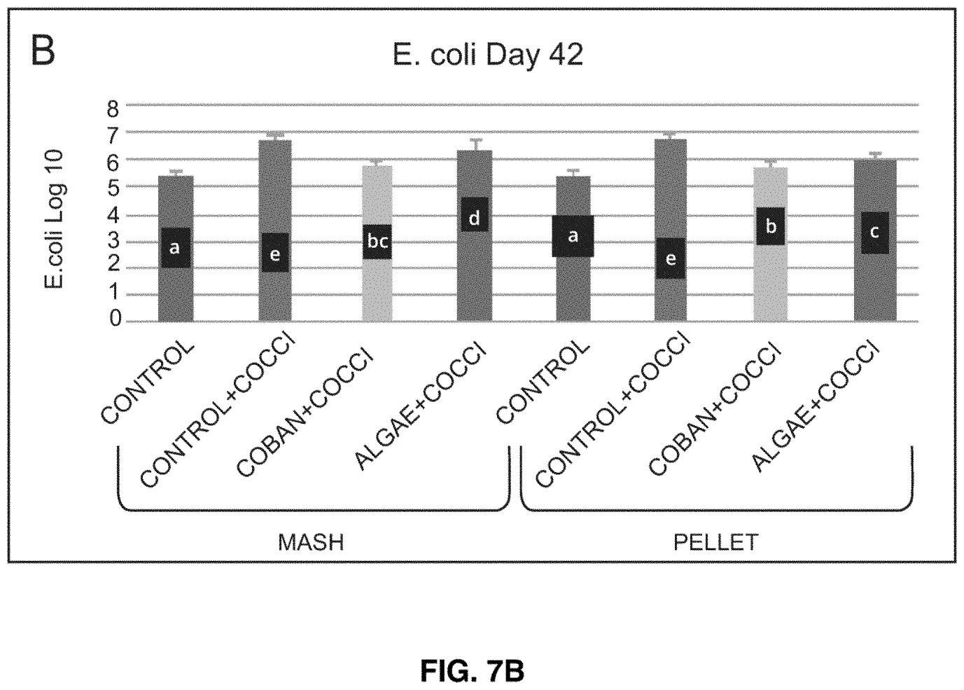

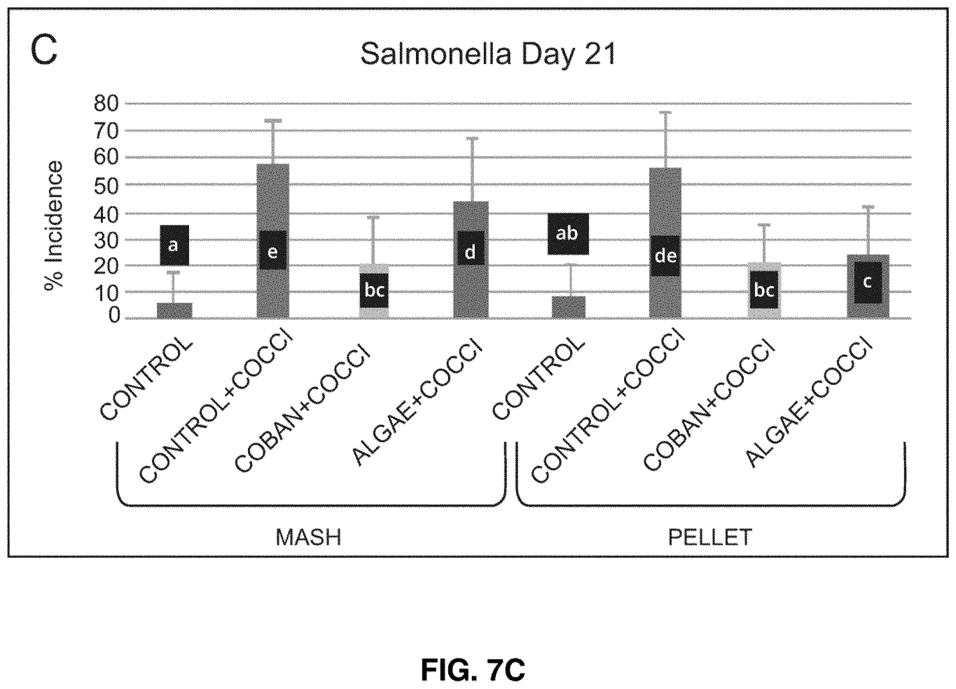

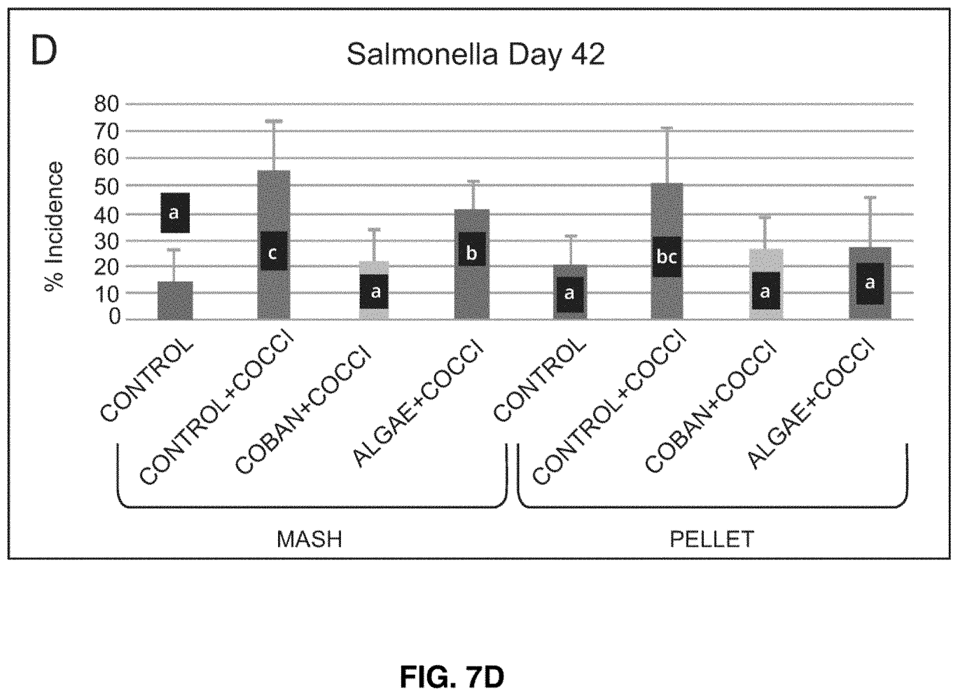

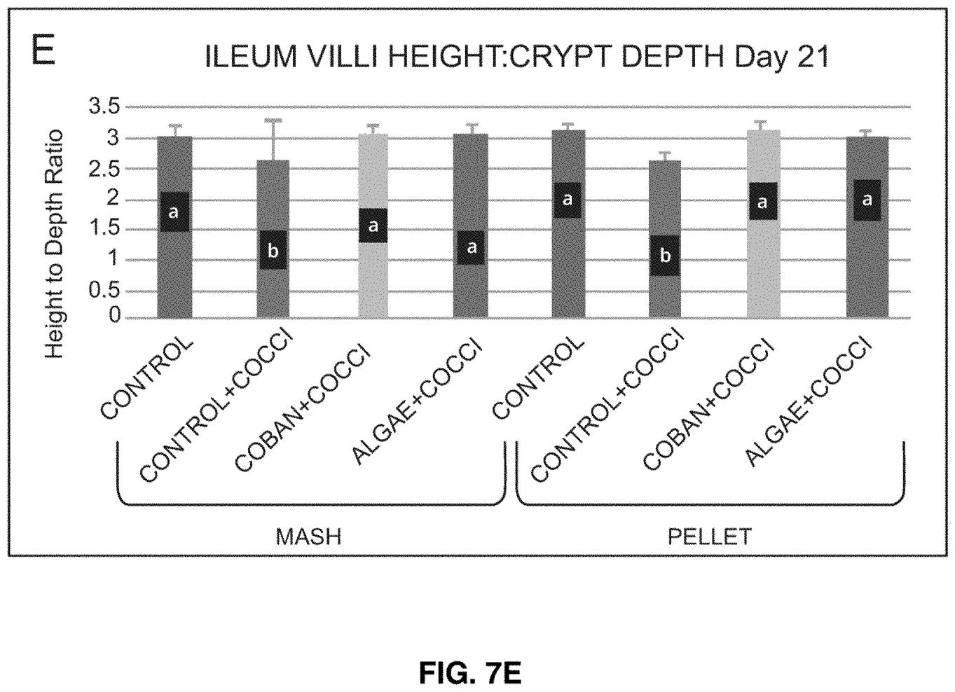

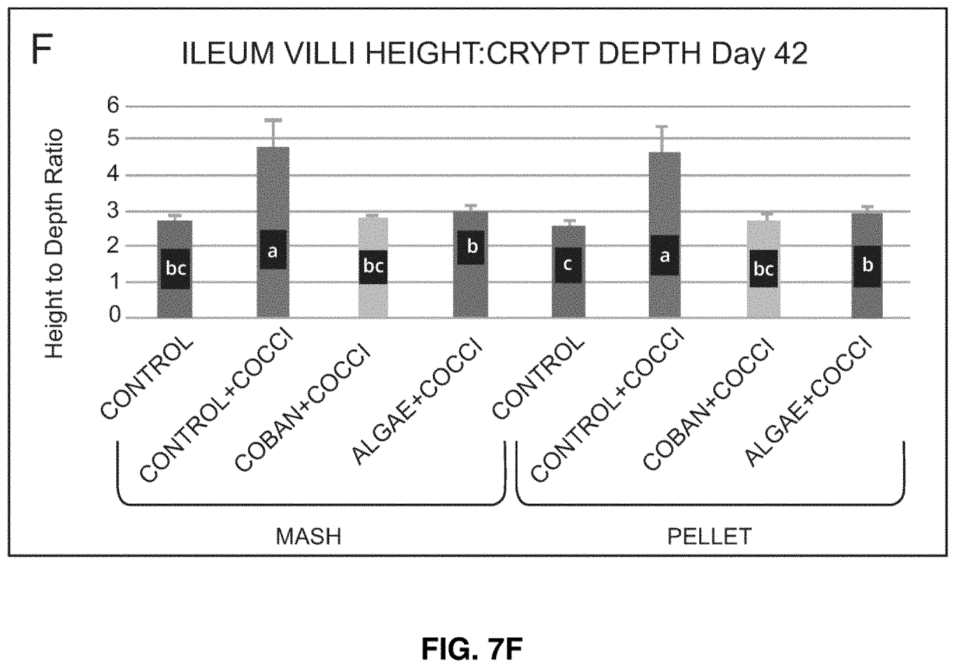

Graphs—Ileum Villi at Days 21 and 42—As illustrated in FIGS. 7A and 7B, ileum villi cell height was significantly increased in the ALGAE+COCCI group compared to the CONTROL+COCCI group on Days 21 and 42 respectively in both the MASH and PELLET fed segments. There were no significant differences in villi cell height in the ALGAE+COCCI and CONTROL groups on Day 21 in the MASH fed segment. However, the ALGAE+COCCI group did not perform as well as the COBAN+COCCI or CONTROL groups on Day 21 in the PELLET fed segment or on Day 42 regardless of feed type. Ileum crypt depth was significantly increased in the ALGAE+COCCI group compared to the CONTROL+COCCI group on both Day 21 and 42 in both the MASH and PELLET fed segments. On Day 21, there were no significant differences in the ALGAE+COCCI group and CONTROL group in either MASH or PELLET fed segments. However, this did not hold true in either segment on Day 42.

As illustrated in FIGS. 7C and 7D, the villi height to crypt depth ratio was significantly decreased in the ALGAE+COCCI group compared to the CONTROL+COCCI group on both Day 21 and 42 respectively in both the MASH and PELLET fed segments.

As shown in FIGS. 7E and 7F, the ileum villi height-to-depth ratio of the coccidia-challenged animals at Days 21 and 42 respectively demonstrates the morphological results of a compromised ability to absorb nutrients brought on by disease. The remaining conditions demonstrate a healthy gut having a relatively high absorptive surface area for nutrients with the birds having been treated with the disclosed treatment compound showing a villi height-to-depth ratio almost the same as those without being subject to the coccidia challenge as well as those to which an anti-coccidia treatment was administered but without the accompanying side effects.

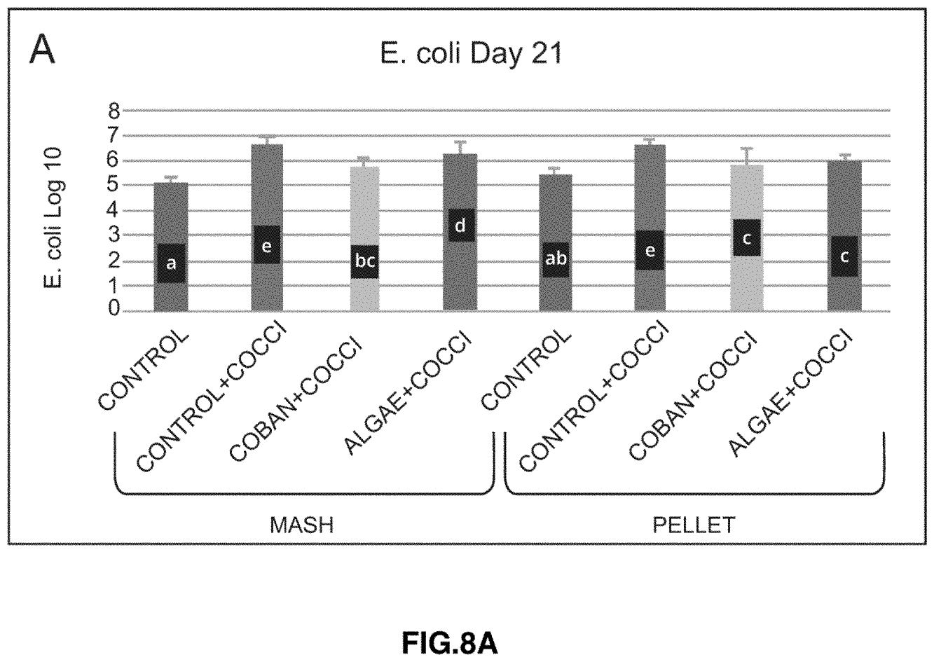

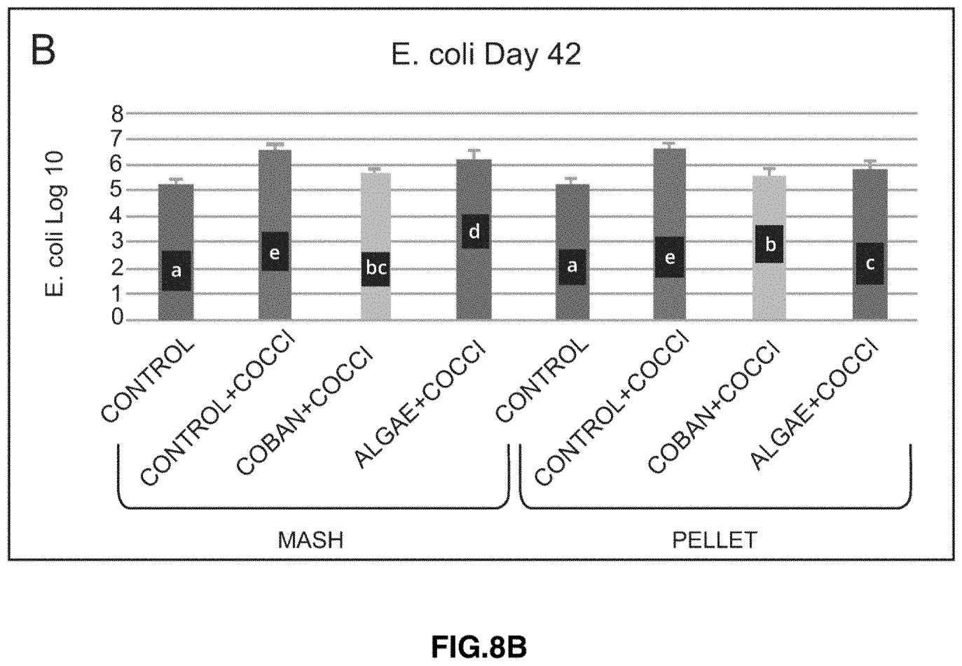

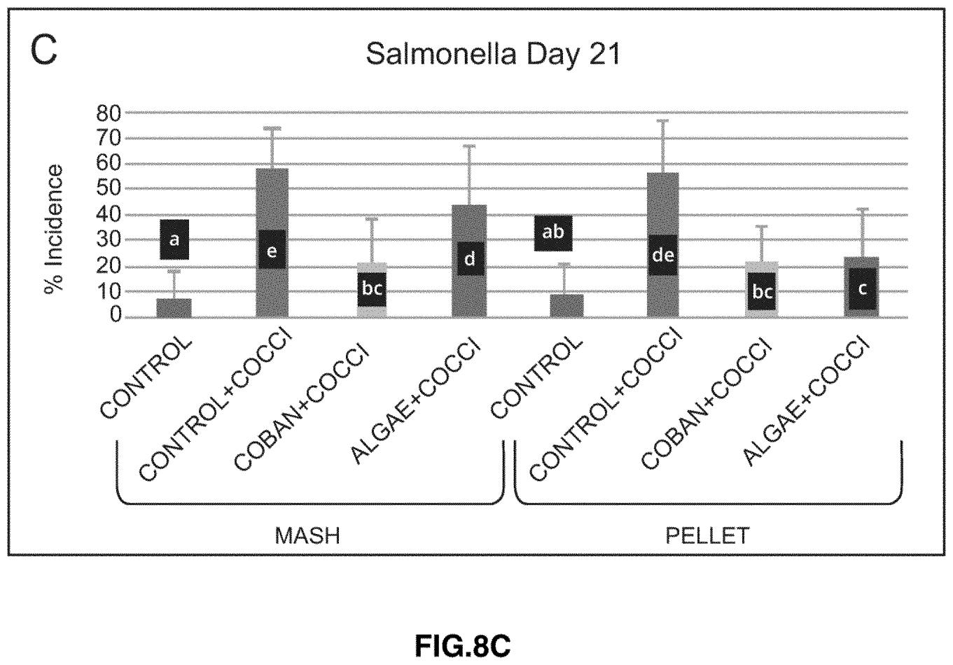

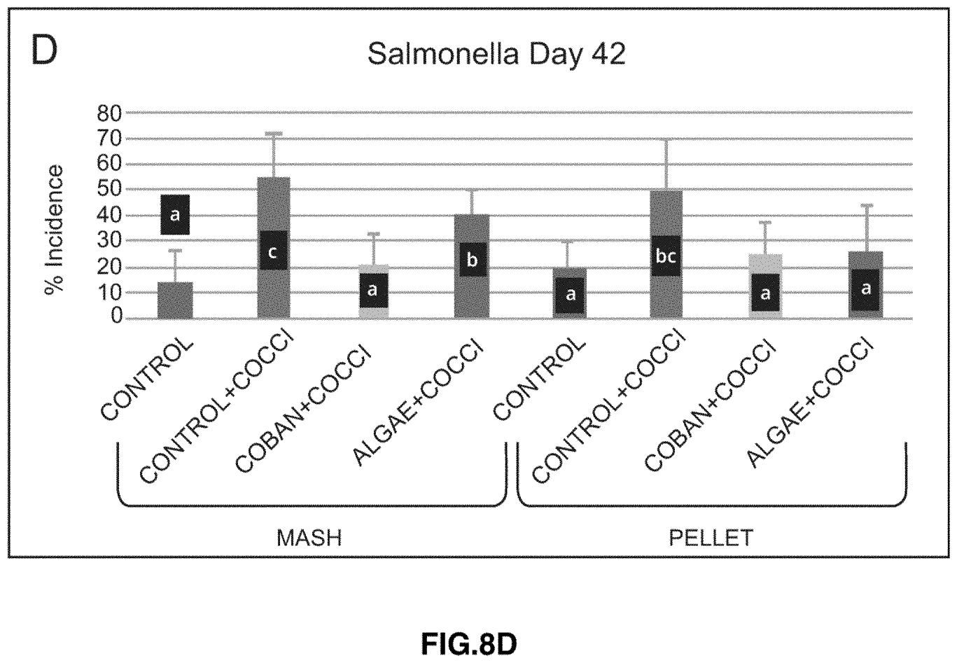

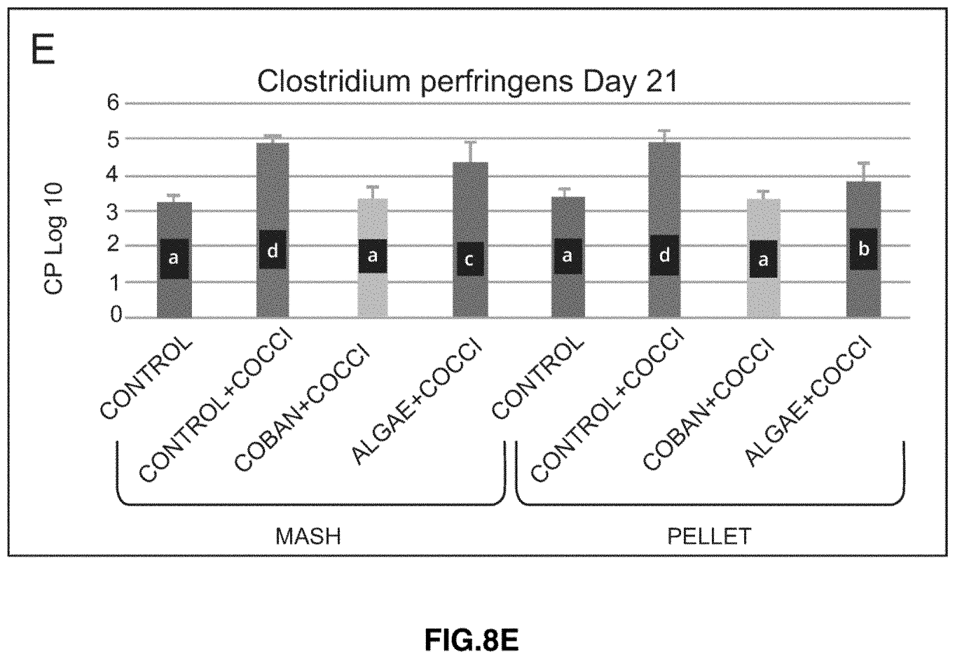

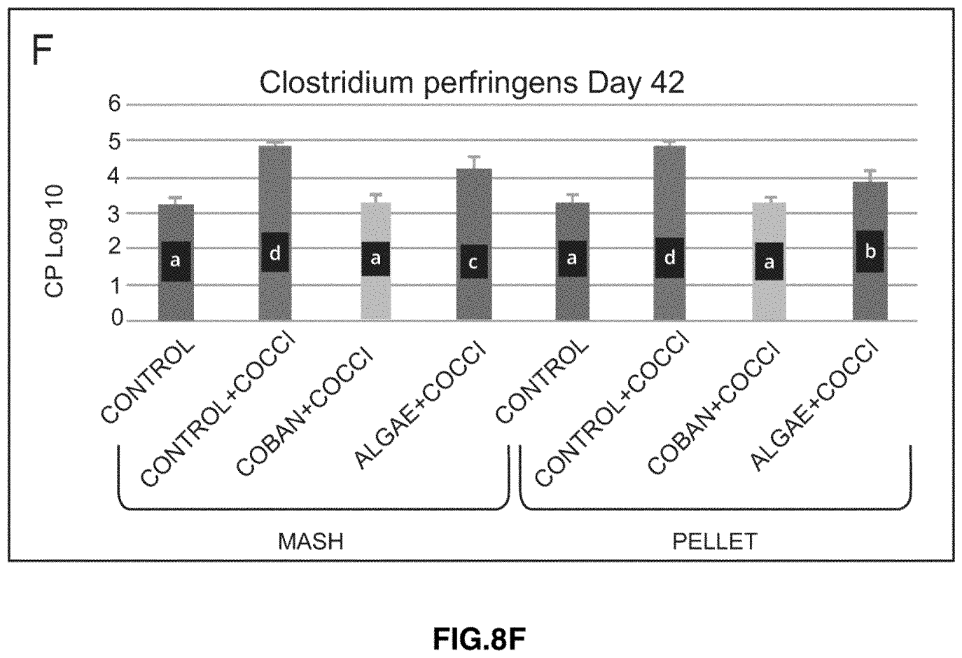

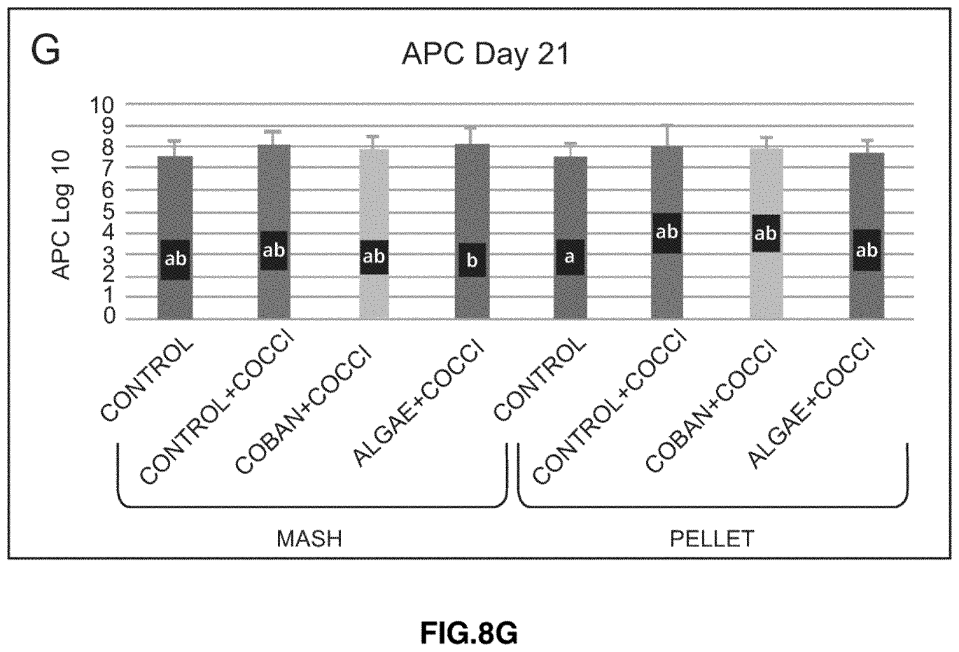

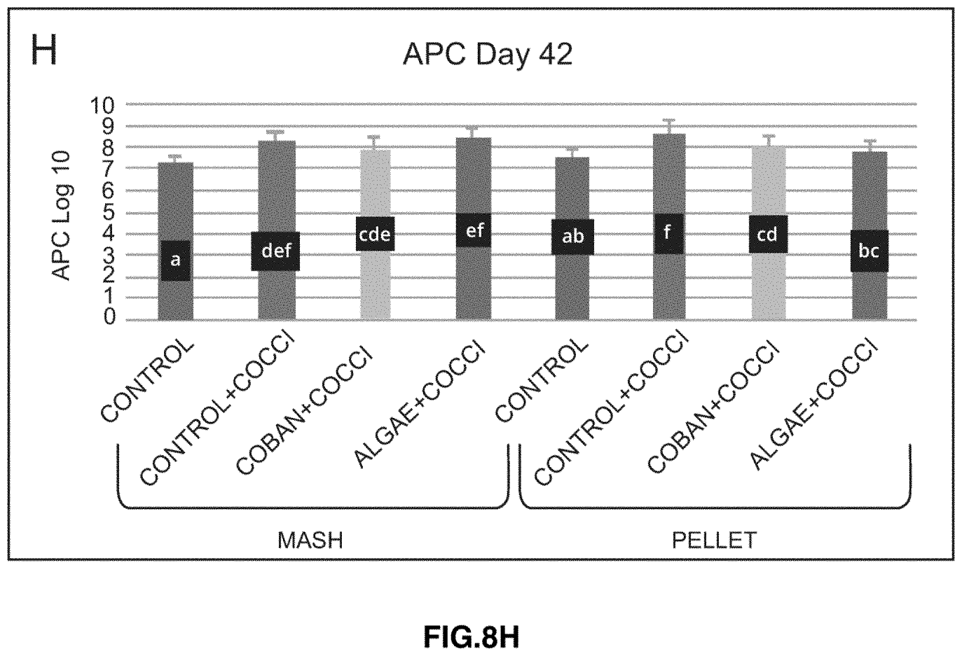

Graphs—Bacteria—As noted above, coccidiosis damages the gut of the animal, thus often acting as a predisposing factor to the rapid onset of bacterial infection and consequential disease, such as necrotic enteritis. Intestinal fecal samples were collected on Days 21 and 42 for incidence of E. coli (FIGS. 8A and 8B) and Salmonella (FIGS. 8C and 8D), analysis of C. perfringens count (FIGS. 8E and 8F), and total aerobic plate count (APC) (FIGS. 8G and 8H). As illustrated in FIGS. 8A-8F, on both Days 21 and 42, the incidences of E. coli, Salmonella, and C. perfringens were significantly decreased in the ALGAE+COCCI group compared to the CONTROL+COCCI group in both the MASH and PELLET fed segments. There were no differences in these two groups in terms of APC, except for on Day 42 when the PELLET fed ALGAE+COCCI group had a significantly lower APC than the CONTROL+COCCI group. On Day 21, there was no difference in the count and incidence of E. coli and Salmonella in the ALGAE+COCCI compared to COBAN+COCCI in the PELLET fed segment. On Day 42, there was no significant difference in Salmonella incidence in the ALGAE+COCCI group compared to CONTROL in the PELLET segment. On Day 42, the APC of the ALGAE+COCCI group was not statistically different from the CONTROL and COBAN+COCCI groups in the PELLET fed segment.

Results

The cocci-challenge was successfully employed as evidenced by the fact that the CONTROL group out-performed the CONTROL+COCCI group across live performance and digestive health parameters for weight gain, feed efficiency, mortality, intestinal villi cell height, crypt depth, villi height to crypt depth ratio, and intestinal coccidiosis across all age ranges studied (Days 0-14, 0-28, 0-35, and 0-42) in both the MASH and PELLET fed segments. The Coccidia Check Score of the CONTROL+COCCI group on Day 21 (2.96 in both the MASH and PELLET fed segments) indicate a ranking of severe coccidiosis. Whereas, mortality rates of 8-9% (Days 0-42) for the CONTROL+COCCI group indicated that a mild to moderate disease challenge was achieved. Poultry industry mortality is typically <4.5% when birds are grown on built-up litter bedding floors, as compared to >11% for a high disease challenge model.

The results demonstrate that pelleted feed was equal or superior to mash in all measured parameters in all groups. These results are consistent with those seen in practice in the poultry industry, which routinely (>90%) utilizes pellet feed for exactly this reason, among others, such as flowability and potential sterilization factors. Based on these results, it can be concluded that that the pelleting process does not negatively affect the test feed ingredient.

The disclosed inventive treatment compound is effective in ameliorating the physical effects of environmental stress on live performance that are typically experienced in poultry production. Over the course of the study (Days 0-42), the PELLET fed ALGAE+COCCI group performed as well as CONTROL and COBAN+COCCI groups on the live performance parameters: body weight gain, feed consumption, FCR, and mortality. In addition, the disclosed inventive treatment compound helped reduce lesion scores to the same degree as seen in the COBAN+COCCI group and reduced the Coccidia Check Score to equal or better than the COBAN+COCCI group. The disclosed inventive treatment compound also reduced the incidence of Salmonella in the gut to the same degree as found in CONTROL and COBAN+COCCI groups. Finally, disclosed inventive treatment compound performs better than CONTROL+COCCI in terms of E. coli and C. perfringens levels in the gut.

The disclosed inventive treatment compound demonstrates a positive impact on the gut health of boilers through improved gut morphology under disease stress by improving nutrient uptake and eventual bird growth and overall improved gut integrity. The treatment method and compound may have benefits that go beyond the poultry industry to other animals and possibly to humans.

What is claimed is:

1. An bacterial-based composition to improve gut morphology in an animal by immune priming beginning in its earliest stage of development, the composition comprising an effective amount of a feed ingredient including a lipopolysaccharide derived from Gram-negative bacteria.

2. The composition of claim 1 wherein said Gram-negative bacteria is a member of the group Variovorax.

3. The composition of claim 2 wherein said member of the group Variovorax is Variovorax paradoxus.

4. The composition of claim 1 wherein the composition is for the treatment of disease in poultry.

5. The composition of claim 4 wherein the composition is for the treatment of parasitic disease in poultry.

6. The composition of claim 5 wherein the composition is for the treatment of coccidiosis in poultry.

7. The composition of claim 4 wherein the composition is for the treatment of bacterial disease in poultry.

8. The composition of claim 7 wherein the composition is for the treatment of necrotic enteritis in poultry.

9. An bacterial-based composition to improve gut morphology in an animal by immune modulation, the composition comprising an effective amount of a biomass-based feed ingredient derived from a Gram-negative bacteria.

10. The composition of claim 9 wherein said Gram-negative bacteria is a member of the group Variovorax.

11. The composition of claim 10 wherein said member of the group Variovorax is Variovorax paradoxus.

12. The composition of claim 9 wherein the composition is for the treatment of parasitic and bacterial disease in poultry.

13. A method for improving gut morphology in an animal, the method comprising the steps of:
forming a biomass-based compound derived from a Gram-negative bacteria at amounts efficacious for the prevention and treatment of disease in the animal;
mixing the compound with an amount of basal feed to create a pre-mix;
adding the premix to finished feed; and
administering the finished feed to the animal from the earliest stage of development.

14. The method of claim 13, including the step of adding an amount of soy oil to the basal feed to create the pre-mix.

15. The method of claim 13 wherein the premix is administered at the concentration range of between about 0.5 lbs. per ton and about 11.0 lbs. per ton of finished feed.

16. The method of claim 13 wherein the premix is administered at the concentration range of between about 3.5 lbs. per ton of finished feed.

17. The method of claim 13 wherein the biomass-based compound derived from a Gram-negative bacteria is a lipopolysaccharide.

18. The method of claim 17 wherein said Gram-negative bacteria-derived lipopolysaccharide is an agonist of the TLR pathway.

19. The method of claim 17 wherein said Gram-negative bacteria is a member of the group Variovorax.

20. The method of claim 19 wherein said member of the group Variovorax is Variovorax paradoxus.
